(19) United States
(12) Patent Application Publication
(10) Pub. No.: US 2025/0107773 A1
(43) Pub. Date: Apr. 3, 2025

(54) ULTRASOUND PROBE

(71) Applicant: FUJIFILM Corporation, Tokyo (JP)

(72) Inventor: Takatoshi HASE, Ashigarakami-gun (JP)

(73) Assignee: FUJIFILM Corporation, Tokyo (JP)

(21) Appl. No.: 18/813,808

(22) Filed: Aug. 23, 2024

(30) Foreign Application Priority Data
Sep. 28, 2023 (JP) 2023-167790

(51) Int. Cl. A61B 8/00; B06B 1/06

(52) U.S. Cl. CPC A61B 8/4488; B06B 1/0622; B06B 2201/76

(57) ABSTRACT

Provided is an ultrasound probe that can generate a high-definition ultrasound image while suppressing an increase in the number of signal lines for driving.

Each of a plurality of piezoelectric elements arranged in an azimuth direction is divided into a plurality of divisional element portions, which are four or more and ten or less divisional element portions, in an elevation direction, a difference between a maximum value and a minimum value of an aspect ratio represented by a ratio of a thickness with respect to a length of each of the divisional element portions in the elevation direction is within a range of 10% of an average value of aspect ratios of the plurality of divisional element portions, an arrangement pitch of the plurality of divisional element portions in the elevation direction is larger than a wavelength of an ultrasound wave determined by a center frequency of the ultrasound probe, and signal electrode layers of at least two divisional element portions that are disposed at a center in the elevation direction and that are adjacent to each other, among the plurality of divisional element portions, are electrically connected to each other.

### CROSS-REFERENCE TO RELATED APPLICATIONS

The present application claims priority under 35 U.S.C. § 119 to Japanese Patent Application No. 2023-167790, filed on Sep. 28, 2023. The above application is hereby expressly incorporated by reference, in its entirety, into the present application.

### BACKGROUND OF THE INVENTION

### 1. Field of the Invention

The invention relates to an ultrasound probe and more particularly to an ultrasound probe in which each of a plurality of piezoelectric elements arranged in an azimuth direction is divided into a plurality of divisional element portions in an elevation direction.

### 2. Description of the Related Art

In the related art, in a medical field, an ultrasound diagnostic apparatus using an ultrasound image has been put into practical use. In general, the ultrasound diagnostic apparatus of this type transmits an ultrasound beam from an ultrasound probe toward a subject and receives an ultrasound echo from the subject with the ultrasound probe, and an ultrasound image is generated by electrically processing the reception signal.

As the ultrasound probe, for example, a one-dimensional array type probe in which a plurality of piezoelectric elements are linearly arranged in an azimuth direction is used. By driving the plurality of piezoelectric elements while adjusting delay amounts, the depth of a transmission focal point of an ultrasound beam can be made electronically variable on a tomographic plane along the azimuth direction. However, in an elevation direction, a fixed focal point is often determined by an acoustic lens disposed in a front portion of the probe.

On the other hand, as disclosed in JP1995-131895A (JP-H07-131895A) and JP1991-243099A (JP-H03-243099A), by using a two-dimensional array type probe in which a plurality of piezoelectric elements are two-dimensionally arranged, it is possible to adjust the depth of a transmission focal point of each ultrasound beam on a tomographic plane along any direction.

However, in the two-dimensional array type probe, the number of piezoelectric elements is significantly increased. Accordingly, a large number of signal lines are required to drive the probe, the thickness and the weight of a cable connecting the ultrasound probe and an apparatus main body are increased, and a user who operates an ultrasound diagnostic apparatus is heavily burdened by the rigidification. Further, manufacturing costs of the ultrasound diagnostic apparatus inevitably increase, and a processing circuit is inevitably complicated.

### SUMMARY OF THE INVENTION

In this regard, a probe which is a so-called 1.5-dimensional array type probe, in which each of a plurality of piezoelectric elements arranged in an azimuth direction is divided into approximately 3 to 5 element portions in an elevation direction, is used.

In this 1.5-dimensional array type probe, by dividing the piezoelectric element in the elevation direction, it is possible to reduce the number of signal lines for driving the probe while generating a high-definition ultrasound image regardless of the depth while making the depth of a transmission focal point of an ultrasound beam electronically variable also in the elevation direction.

In such a 1.5-dimensional array type probe, in general, a division width of the piezoelectric element in the elevation direction is determined in consideration of the sensitivity of the probe, a beam shape (F-Number), and the like, so that a difference of several times in an aspect ratio represented by a ratio of a thickness with respect to the length of each divided element portion in the elevation direction occurs in some cases.

In a case where a difference in the aspect ratio occurs between the plurality of element portions arranged in the elevation direction, sound speeds are different from each other. Thus, band shapes of sensitivities in the element portions are different from each other, and there is a possibility that a high-definition ultrasound image cannot be generated.

The present invention is devised in order to solve such problems of the related art, and an object thereof is to provide an ultrasound probe that can generate a high-definition ultrasound image while suppressing an increase in the number of signal lines for driving. According to the following configuration, the above object can be achieved.
[1] An ultrasound probe in which a plurality of piezoelectric elements are arranged in an array along an azimuth direction on a backing material,
in which each of the plurality of piezoelectric elements consists of a laminate in which a signal electrode layer, a piezoelectric portion, and a ground electrode layer are laminated in turn on a surface of the backing material,
each of the piezoelectric elements is divided into a plurality of divisional element portions, which are four or more and ten or less divisional element portions, in an elevation direction,
a difference between a maximum value and a minimum value of an aspect ratio represented by a ratio of a thickness with respect to a length of each of the plurality of divisional element portions in the elevation direction is within a range of 10% of an average value of aspect ratios of the plurality of divisional element portions,
an arrangement pitch of the plurality of divisional element portions in the elevation direction is larger than a wavelength of an ultrasound wave determined by a center frequency of the ultrasound probe, and
the signal electrode layers of at least two divisional element portions that are disposed at a center in the elevation direction and that are adjacent to each other, among the plurality of divisional element portions, are electrically connected to each other.
[2] The ultrasound probe according to [1],
in which a wiring board having a conductive pattern is disposed between the plurality of piezoelectric elements and the backing material, and
each of the signal electrode layers and the ground electrode layers of the plurality of piezoelectric elements is led out via the conductive pattern of the wiring board.
[3] The ultrasound probe according to [2],
in which each of the plurality of divisional element portions is split in the elevation direction via a split groove extending from the ground electrode layer to the signal electrode layer.
[4] The ultrasound probe according to [3],
in which the split groove extends into the wiring board.
[5] The ultrasound probe according to [3] or [4],
in which an acoustic matching layer is laminated on the ground electrode layers of the plurality of piezoelectric elements.
[6] The ultrasound probe according to [5],
in which the split groove extends into the acoustic matching layer.
[7] The ultrasound probe according to any one of [3] to [6],
in which the split groove has a groove width of 10 um or more and 30 um or less in the elevation direction.
[8] The ultrasound probe according to [7],
in which the split groove is filled with a filler.
[9] The ultrasound probe according to [2],
in which the at least two divisional element portions are split in the elevation direction in terms of mechanical vibration while each being connected to a part of the piezoelectric portion and the signal electrode layer.
[10] The ultrasound probe according to [9],
in which the piezoelectric portion has a first surface that is in contact with the signal electrode layer and a second surface that is in contact with the ground electrode layer, and
the piezoelectric portions of the at least two divisional element portions are adjacent to each other in the elevation direction via a divisional groove extending from the second surface toward the first surface.
[11] The ultrasound probe according to [10],
in which the divisional groove has a depth dimension larger than 90% of a thickness of the piezoelectric portion.
[12] The ultrasound probe according to or [11],
in which the divisional groove has a groove width of 10 μm or more and 30 μm or less in the elevation direction.
[13] The ultrasound probe according to [12],
in which the divisional groove is filled with a filler.

With the present invention, each of the plurality of piezoelectric elements consists of the laminate in which the signal electrode layer, the piezoelectric portion, and the ground electrode layer are laminated in turn on the surface of the backing material, each of the plurality of piezoelectric elements is divided into four or more divisional element portions in the elevation direction, the difference between the maximum value and the minimum value of the aspect ratio represented by the ratio of the thickness with respect to the length of each of the plurality of divisional element portions in the elevation direction is within the range of 10% of the average value of the aspect ratios of the plurality of divisional element portions, the arrangement pitch of the plurality of divisional element portions in the elevation direction is larger than the wavelength of the ultrasound wave determined by the center frequency of the ultrasound probe, and the signal electrode layers of at least two divisional element portions that are disposed at the center in the elevation direction and that are adjacent to each other, among the plurality of divisional element portions, are electrically connected to each other. Therefore, it is possible to generate a high-definition ultrasound image while suppressing an increase in the number of signal lines for driving.

### BRIEF DESCRIPTION OF THE DRAWINGS

FIG. 1 is a partial perspective view showing an ultrasound probe according to embodiment 1 of the present invention.

FIG. 2 is a cross-sectional view taken along an elevation direction of the ultrasound probe according to embodiment 1 of the present invention.

FIG. 3 is a graph showing a simulation result of calculating a frequency band of a sensitivity in a case where an aspect ratio of a piezoelectric element is changed.

FIG. 4 is a flowchart showing a manufacturing method of the ultrasound probe according to embodiment 1 of the present invention.

FIG. 5 is a cross-sectional view taken along an elevation direction of an ultrasound probe according to embodiment 2 of the present invention.

FIG. 6 is a cross-sectional view taken along an elevation direction of an ultrasound probe according to embodiment 3 of the present invention.

FIG. 7 is a cross-sectional view taken along an elevation direction of an ultrasound probe according to embodiment 4 of the present invention.

FIG. 8 is a cross-sectional view taken along an elevation direction of an ultrasound probe according to embodiment 5 of the present invention.

### DESCRIPTION OF THE PREFERRED EMBODIMENTS

Hereinafter, embodiments of the invention will be described with reference to the accompanying drawings.

Description of configuration requirements written below is provided based on representative embodiments of the present invention, but the present invention is not limited to such embodiments.

In the present specification, a numerical range represented using “to” means a range including numerical values before and after “to” as a lower limit value and an upper limit value.

In the present specification, “same” and “identical” include an error range generally allowed in the technical field.

### Embodiment 1

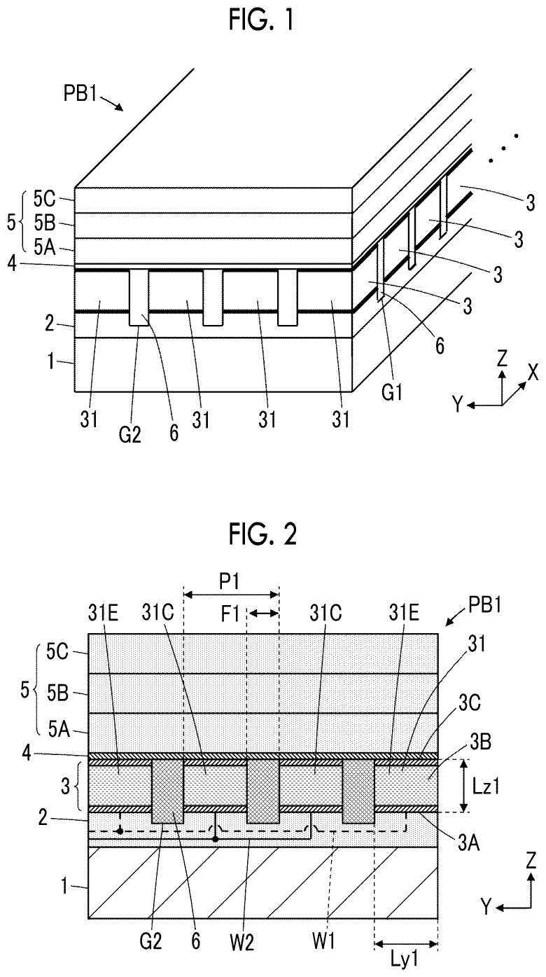

FIG. 1 shows a configuration of an ultrasound probe PB1 according to embodiment 1 of the present invention. The ultrasound probe PB1 has a configuration where a backing material 1, a wiring board 2, a plurality of piezoelectric elements 3, a conductive layer 4, and an acoustic matching layer 5 are laminated in turn in a lamination direction.

The backing material 1 is a flat plate-shaped member that extends over the entire length direction and the entire width direction of the ultrasound probe PB1, and the wiring board 2 extends on a surface of the backing material 1 and over the entire surface of the backing material 1.

The plurality of piezoelectric elements 3 disposed on the wiring board 2 are arranged in an array along an azimuth direction, and a split groove G1 extending in each of an elevation direction and the lamination direction is formed between the piezoelectric elements 3 adjacent to each other, and the split groove G1 is filled with a filler 6. In addition, each of the plurality of piezoelectric elements 3 that are arranged in the azimuth direction is divided into four divisional element portions 31 in the elevation direction, and between the divisional element portions 31 adjacent to each other, a split groove G2 extending in each of the azimuth direction and the lamination direction is formed, and the split groove G2 is filled with the filler 6.

Herein, for convenience, the azimuth direction in which the flat plate-shaped backing material 1 extends along an XY-plane and the plurality of piezoelectric elements 3 are arranged is called an X-direction, the elevation direction in which each of the piezoelectric elements 3 is divided into the four divisional element portions 31 is called a Y-direction, and the lamination direction from the backing material 1 toward the acoustic matching layer 5 is called a +Z-direction.

The conductive layer 4 extending along the XY-plane is disposed on the plurality of piezoelectric elements 3, and the acoustic matching layer 5 extending along the XY-plane is further disposed on the conductive layer 4.

The acoustic matching layer 5 has a structure in which a first acoustic matching layer 5A, a second acoustic matching layer 5B, and a third acoustic matching layer 5C are laminated in turn on the conductive layer 4.

The split grooves G1 and G2 in the plurality of the piezoelectric elements 3 both extend from a boundary portion between the piezoelectric element 3 and the conductive layer 4 into the wiring board 2 in a Z-direction.

The backing material 1 supports the plurality of piezoelectric elements 3 via the wiring board 2, absorbs ultrasound waves radiated from the plurality of piezoelectric elements 3 in a −Z-direction, which is a rear side of the ultrasound probe PB1, and is formed of a rubber material such as ferrite rubber.

The wiring board 2 disposed on the backing material 1 consists of, for example, a flexible printed circuit (FPC) and has a plurality of conductive patterns (not shown) corresponding to all the divisional element portions 31, which are four divisional element portions divided from each of the plurality of piezoelectric elements 3 in the elevation direction.

The conductive layer 4 disposed on the plurality of piezoelectric elements 3 forms a common ground potential layer for all the divisional element portions 31.

The acoustic matching layer 5 is a layer for matching acoustic impedance between the piezoelectric elements 3 and a subject so that it is easy to make the ultrasound waves incident into the subject. The acoustic matching layer 5 can be formed of a material that has acoustic impedance having a value lower than the acoustic impedance of the piezoelectric elements 3 and higher than the acoustic impedance of the subject. For example, by setting the acoustic impedance of the second acoustic matching layer 5B to be lower than the acoustic impedance of the first acoustic matching layer 5A and by setting the acoustic impedance of the third acoustic matching layer 5C to be lower than the acoustic impedance of the second acoustic matching layer 5B, a layer structure in which the acoustic impedance gradually decreases from the piezoelectric elements 3 toward the subject is formed.

The acoustic matching layer 5 is not limited to being composed of three layers including the first acoustic matching layer 5A, the second acoustic matching layer 5B, and the third acoustic matching layer 5C and can be composed of two or less layers or four or more layers.

Since the fillers 6 filling the split grooves G1 and G2 are for fixing positions and postures of the piezoelectric elements 3 adjacent to each other in the X-direction and positions and postures of the divisional element portions 31 adjacent to each other in the Y-direction, for example, an epoxy resin, a silicone rubber, or the like can be used as the fillers 6.

As shown in FIG. 2, the piezoelectric element 3 is formed of a laminate in which a signal electrode layer 3A, a piezoelectric portion 3B, and a ground electrode layer 3C are laminated in turn on the wiring board 2.

The piezoelectric portion 3B is formed of a known piezoelectric material, emits ultrasound waves by expanding and contracting in response to application of a voltage, and generates an electric signal by expanding and contracting in response to receipt of a so-called ultrasound echo from the outside. Examples of the piezoelectric material include piezoelectric ceramics represented by lead zirconate titanate (PZT), polymer piezoelectric elements represented by poly vinylidene di fluoride (PVDF), and piezoelectric single crystals represented by lead magnesium niobate-lead titanate (PMN-PT).

Since the signal electrode layer 3A is used in order to apply a voltage to the piezoelectric portion 3B and in order to extract an electric signal generated in response to the expansion and contraction of the piezoelectric portion 3B by receiving a so-called ultrasound echo.

The ground electrode layer 3C is used in order to set a reference potential of the piezoelectric portion 3B to a ground potential.

The signal electrode layer 3A and the ground electrode layer 3C is composed of a metal thin film formed on a surface of the piezoelectric portion 3B on a −Z-direction side and a surface of the piezoelectric portion 3B on a +Z direction side, respectively, using, for example, a sputtering method.

The four divisional element portions 31 divided in the elevation direction have substantially the same aspect ratio R as each other. Herein, the aspect ratio R is represented by a ratio of a thickness Lz1 in the Z-direction with respect to a length Ly1 of each of the divisional element portions 31 in the elevation direction. Specifically, a difference dR between a maximum value Rmax and a minimum value Rmin of the aspect ratios R of the four divisional element portions 31 divided in the elevation direction is set to be within a range of 10% of an average value Rav of the aspect ratios R of the four divisional element portions 31.

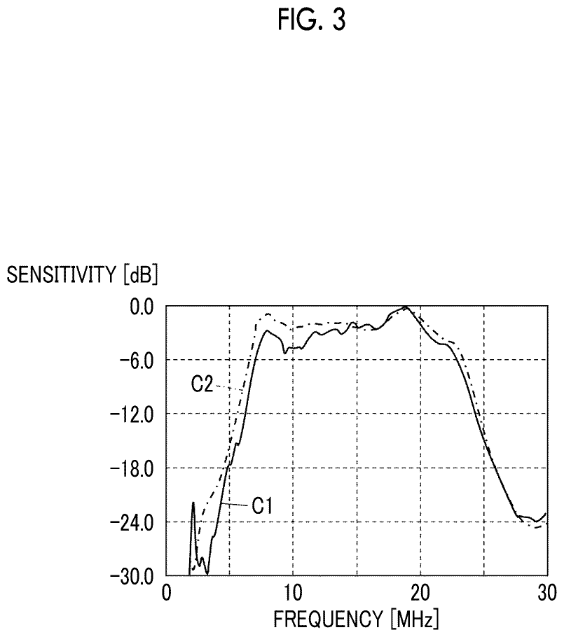

FIG. 3 shows a simulation result of calculating a frequency band of a sensitivity of a piezoelectric element in a case where the aspect ratio R of the piezoelectric element is changed. In FIG. 3, a curve C1 shows a band shape of a piezoelectric element having the aspect ratio R=0.05, and a curve C2 shows a band shape of a piezoelectric element having the aspect ratio R=0.025.

As shown in FIG. 3, in a case where the aspect ratios R of the piezoelectric elements are different, sound speeds in the piezoelectric elements are different. Thus, a difference in the frequency band shape of the sensitivity is generated. Therefore, in a case where there is a difference having a degree determined in advance or more in the aspect ratios R of the four divisional element portions 31 divided in the elevation direction, there is a risk that a high-definition ultrasound image along the elevation direction cannot be generated.

However, in the ultrasound probe PB1 according to embodiment 1, since the difference dR between the maximum value Rmax and the minimum value Rmin of the aspect ratios R of the four divisional element portions 31 divided in the elevation direction is set to be within the range of 10% of the average value Rav of the aspect ratios R of the four divisional element portions 31, the frequency band shapes of sensitivities in the four divisional element portions 31 are substantially the same, and it is possible to generate a high-definition ultrasound image along the elevation direction.

In order to further improve the resolution of the ultrasound image along the elevation direction by making the frequency band shapes of the sensitivities in the four divisional element portions 31 more similar to each other, it is preferable that the difference dR between the maximum value Rmax and the minimum value Rmin of the aspect ratios R of the four divisional element portions 31 is set to be, for example, within a range of 5% of the average value Rav of the aspect ratios R of the four divisional element portions 31. By designing the ultrasound probe PB1 such that the four divisional element portions 31 have the same aspect ratio R as each other and manufacturing the ultrasound probe PB1 while suppressing a manufacturing tolerance to, for example, within 5%, the difference dR between the maximum value Rmax and the minimum value Rmin of the aspect ratios R can be set within the range of 5% of the average value Rav.

In addition, as shown in FIG. 2, among the four divisional element portions 31 divided in the elevation direction, the signal electrode layers 3A of divisional element portions 31E at two end parts disposed at both ends in the elevation direction are connected to one conductive pattern W1 of the wiring board 2, and the two divisional element portions 31E are configured to be simultaneously driven by a common drive voltage via the conductive pattern W1.

Further, among the four divisional element portions 31 divided in the elevation direction, the signal electrode layers 3A of two divisional element portions 31C positioned at the center are both connected to a conductive pattern W2 different from the conductive pattern W1 of the wiring board 2, and the two divisional element portions 31C are configured to be simultaneously driven by a common drive voltage via the conductive pattern W2.

By simultaneously driving the two divisional element portions 31C at the center with adjustment of delay amounts with respect to the divisional element portions 31E at the two end parts, the depth of a transmission focal point of an ultrasound beam can be made electronically variable on a tomographic plane along the elevation direction.

As described above, in the ultrasound probe PB1 according to embodiment 1, the depth of the transmission focal point of the ultrasound beam is electronically variable in the elevation direction, but the ultrasound beam is not steered in the elevation direction.

Herein, it is known that in a case where an ultrasound beam is steered at an angle θ, in order to suppress the occurrence of so-called grating lobes, it is necessary to define a wavelength of an ultrasound wave emitted from the ultrasound probe PB1 as λ and define an arrangement pitch P of the piezoelectric elements arranged in an array as P<λ/(1+|sin θ|).

That is, it is necessary to set the arrangement pitch P of the piezoelectric elements to be smaller than the wavelength of the ultrasound wave. For example, in a case where the steering is performed at an angle of 45 degrees, P<0.6λ is satisfied.

In general, in a two-dimensional array type probe, since an ultrasound beam is steered in both the azimuth direction and the elevation direction, it is necessary to make both an arrangement pitch of the piezoelectric elements in the azimuth direction and an arrangement pitch of the piezoelectric elements in the elevation direction smaller than a wavelength of an ultrasound wave emitted from the probe.

In the ultrasound probe PB1 according to embodiment 1, it is desirable that the arrangement pitch of the piezoelectric elements 3 in the azimuth direction is set to a value smaller than the wavelength of the ultrasound wave in order to enable the steering of the ultrasound beam while suppressing the occurrence of the grating lobes in the tomographic plane along the azimuth direction.

However, in the ultrasound probe PB1 according to embodiment 1, since an ultrasound beam is not steered in the elevation direction, it is not necessary to suppress the occurrence of grating lobes. For this reason, the arrangement pitch P1 of the four divisional element portions 31 in the elevation direction is set to a value larger than a wavelength determined by a center frequency of an ultrasound wave emitted from the ultrasound probe PB1.

Accordingly, for example, in terms of the division of the four divisional element portions 31 in the elevation direction, the registration of the divisional element portions 31 and the conductive patterns W1 and W2 of the wiring board 2, and the like, it is possible to easily manufacture the ultrasound probe PB1 compared to the two-dimensional array type probe.

In addition, the split groove G2 that splits the divisional element portions 31 adjacent to each other has a groove width F1 of 10 μm or more and 30 μm or less, preferably 20 μm or less in the elevation direction.

By making the groove width F1 of the split groove G2 small, the divisional element portions 31 that are long in the elevation direction can be secured in a case where a total length of the ultrasound probe PB1 in the elevation direction is kept constant, and the sensitivity of the ultrasound probe PB1 can be improved.

The divisional element portion 31 in the ultrasound probe PB1 according to embodiment 1 is formed, for example, in a size of the length Ly1=1 mm in the elevation direction, the length Lx1=0.07 mm in the azimuth direction, and a thickness Lz1=0.1 mm in the Z-direction.

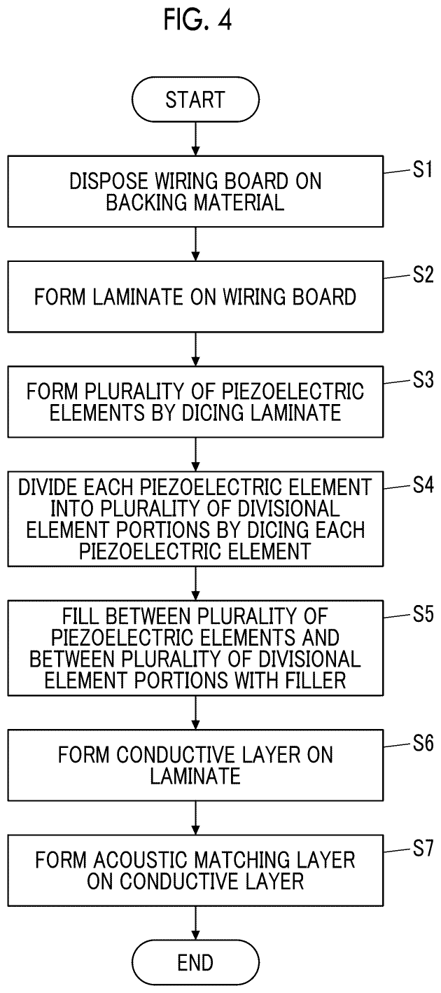

Next, a manufacturing method of the ultrasound probe PB1 according to embodiment 1 will be described with reference to a flowchart of FIG. 4.

First, in step S1, the wiring board 2 is disposed on the surface of the backing material 1. The wiring board 2 is bonded to the backing material 1 with, for example, an adhesive or the like.

Next, in step S2, a laminate in which the signal electrode layer 3A, the piezoelectric portion 3B, and the ground electrode layer 3C are laminated in turn on the wiring board 2 is formed. The signal electrode layer 3A, the piezoelectric portion 3B, and the ground electrode layer 3C are bonded to each other with, for example, an adhesive or the like.

In step S3, the plurality of piezoelectric elements 3 arranged in the X-direction are formed by dicing the laminate to form the plurality of split grooves Gl extending along each of the Y-direction and the Z-direction.

In subsequent step S4, by dicing each piezoelectric element 3 to form the plurality of split grooves G2 extending along the X-direction and the Z-direction, the piezoelectric element 3 is divided into the plurality of divisional element portions 31 arranged in the Y-direction.

Both the split grooves Gl and G2 extend from the ground electrode layer 3C of the piezoelectric element 3 into the wiring board 2 in the Z-direction.

Further, in step S5, the filler 6 fills each of the split groove G1 disposed between the plurality of piezoelectric elements 3 and the split groove G2 disposed between the plurality of divisional element portions 31.

Next, after the conductive layer 4 is formed on the laminate in step S6, the acoustic matching layer 5 is formed on the conductive layer 4 in step S7. Herein, the conductive layer 4 and the acoustic matching layer 5 are disposed to cover all the plurality of piezoelectric elements 3 arranged in the X-direction and the divisional element portions 31 arranged in the Y-direction.

The conductive layer 4 is formed, for example, by attaching a metal foil onto the laminate, and the acoustic matching layer 5 is formed, for example, by bonding the first acoustic matching layer 5A, the second acoustic matching layer 5B, and the third acoustic matching layer 5C in turn at a temperature of, for example, 80° C. to 100° C.

In this manner, as shown in FIG. 1, the ultrasound probe PB1, in which the plurality of piezoelectric elements 3 are arranged in the X-direction, which is the azimuth direction, and each of the piezoelectric elements 3 is divided into the plurality of divisional element portions 31 in the Y-direction, which is the elevation direction, is manufactured.

The plurality of conductive patterns W1 and W2 corresponding to the plurality of piezoelectric elements 3 and the plurality of divisional element portions 31 are formed in advance in the wiring board 2, the laminate is formed on the wiring board 2 in step S2, and the plurality of divisional element portions 31 divided from each piezoelectric element 3 by dicing the laminate in steps S3 and S4 are disposed on the corresponding conductive patterns W1 and W2. Accordingly, the signal electrode layer 3A of each of the divisional element portions 31 is bonded to and electrically connected to the corresponding conductive patterns W1 and W2 of the wiring board 2.

In such a manner, among the four divisional element portions 31 divided in the elevation direction, the signal electrode layers 3A of the divisional element portions 31E at the two end parts disposed at both ends in the elevation direction are both connected to the conductive pattern W1 of the wiring board 2, and the signal electrode layers 3A of the two divisional element portions 31C disposed at the center in the elevation direction are both connected to the conductive pattern W2 of the wiring board 2.

In addition, each of the ground electrode layers 3C of all the divisional element portions 31 in the ultrasound probe PB1 is electrically connected to the conductive layer 4 by being bonded to the conductive layer 4, and the conductive layer 4 is connected to a ground pattern of the wiring board 2 via a resin film with a conductive film (not shown) or the like.

Then, each of the signal electrode layers 3A and the ground electrode layers 3C of all the divisional element portions 31 is led out from the ultrasound probe PB1 via the conductive patterns W1 and W2 of the wiring board 2.

The connection between the signal electrode layer 3A and the ground electrode layer 3C of each of the divisional element portions 31 and the wiring board 2 is not limited to the above method and can be performed through various types of methods using, for example, solder, a conductive resin, a sputtered film, or the like.

By applying a voltage, via the wiring board 2, to each of the signal electrode layers 3A and the ground electrode layers 3C of the four divisional element portions 31 divided in the elevation direction from each of the plurality of piezoelectric elements 3 arranged in the azimuth direction, the depth of the transmission focal point of an ultrasound beam is electronically variable also in the elevation direction, and thus it is possible to generate a high-definition ultrasound image both in a shallow portion and in a deep portion.

In the ultrasound probe PB1 according to embodiment 1, each of the plurality of piezoelectric elements 3 arranged in the azimuth direction is divided into the four divisional element portions 31 in the elevation direction, the signal electrode layers 3A of the divisional element portions 31E at the two end parts disposed at both ends in the elevation direction are electrically connected to each other by the conductive pattern W1 of the wiring board 2, and the signal electrode layers 3A of the two divisional element portions 31C positioned at the center in the elevation direction are electrically connected to each other by the conductive pattern W2 of the wiring board 2. Therefore, it is possible to generate a high-definition ultrasound image both in the shallow portion and in the deep portion by making the depth of the transmission focal point of an ultrasound beam electronically variable also in the elevation direction while suppressing an increase in the number of signal lines for driving.

In the ultrasound probe PB1 of embodiment 1, the divisional element portion 31 has a size of the length Ly1=1 mm in the elevation direction, the length Lx1=0.07 mm in the azimuth direction, and the thickness Lz1=0.1 mm in the Z-direction, but without being limited thereto, can have, for example, an approximate size of the length Ly1=0.5 to 2 mm in the elevation direction, the length Lx1=0.05 to 0.2 mm in the azimuth direction, and the thickness Lz1=0.05 to 0.2 mm in the Z-direction.

### Embodiment 2

In the ultrasound probe PB1 of embodiment 1 shown in FIG. 2, the split grooves G2 separating the four divisional element portions 31 divided in the elevation direction from each other extend from the boundary portion between the piezoelectric element 3 and the conductive layer 4 into the wiring board 2, but the invention is not limited thereto.

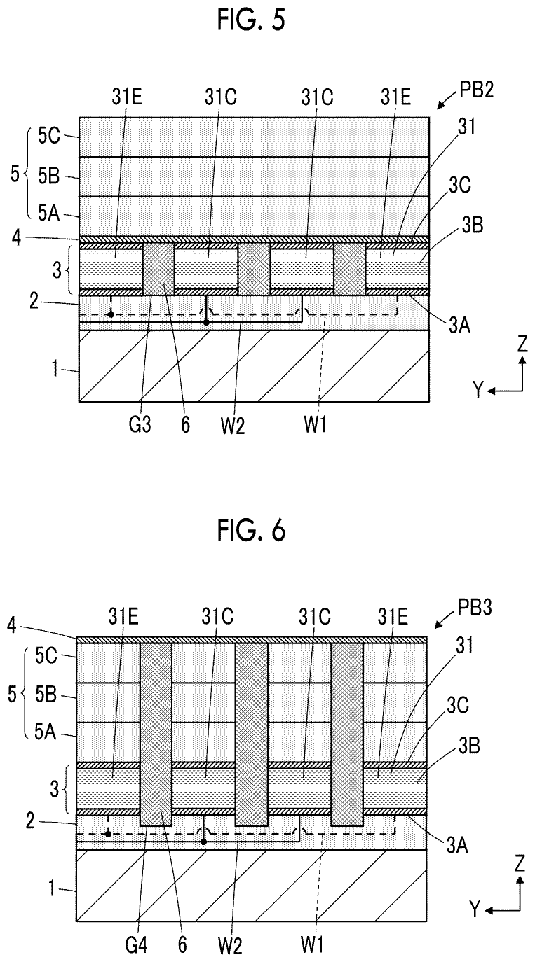

FIG. 5 shows a configuration of an ultrasound probe PB2 according to embodiment 2. In the ultrasound probe PB2, the divisional element portions 31 adjacent to each other in the elevation direction are split from each other by a split groove G3, and the split groove G3 extends from the boundary portion between the piezoelectric element 3 and the conductive layer 4 to a boundary portion between the piezoelectric element 3 and the wiring board 2. That is, the split groove G3 has a groove depth that is the same as the thickness of the piezoelectric element 3 in the Z-direction and does not extend into the wiring board 2.

The split groove G3 is filled with the filler 6.

Although not shown, in the ultrasound probe PB2 according to embodiment 2, like the split groove G3, also a split groove that splits the piezoelectric elements 3 adjacent to each other in the azimuth direction from each other has a groove depth that is the same as the thickness of each piezoelectric element 3 in the Z-direction and does not extend into the wiring board 2.

The ultrasound probe PB2 according to embodiment 2 is obtained by splitting the divisional element portions 31 adjacent to each other in the elevation direction with the split groove G3, instead of the split groove G2, and splitting the piezoelectric elements 3 adjacent to each other in the azimuth direction from each other with the split groove having the groove depth that is the same as the thickness of the piezoelectric element 3 in the Z-direction, instead of the split groove G1 in the ultrasound probe PB1 according to embodiment 1, and the other configurations are the same as those of the ultrasound probe PB1 according to embodiment 1.

Even in the ultrasound probe PB2 having such a configuration, like the ultrasound probe PB1 of embodiment 1, it is possible to generate a high-definition ultrasound image both in the shallow portion and in the deep portion by making the depth of the transmission focal point of an ultrasound beam electronically variable also in the elevation direction while suppressing an increase in the number of signal lines for driving.

### Embodiment 3

FIG. 6 shows a configuration of an ultrasound probe PB3 according to embodiment 3. In the ultrasound probe PB3, a split groove G4 that splits the divisional element portions 31 adjacent to each other in the elevation direction from each other extends to an end part of the acoustic matching layer 5 in the +Z-direction. That is, the split groove G4 extends from the end part of the acoustic matching layer 5 in the +Z-direction into the wiring board 2.

Further, the acoustic matching layer 5 is disposed directly on the ground electrode layer 3C of each of the divisional element portions 31, and the conductive layer 4 extending along the XY-plane is formed on the end part of the acoustic matching layer 5 in the +Z-direction to extend over the entire ultrasound probe PB3.

The ground electrode layer 3C of each of the divisional element portions 31 is electrically connected to the conductive layer 4, for example, via a resin film with a conductive film (not shown) or the like.

The split groove G4 is filled with the filler 6.

Although not shown, in the ultrasound probe PB3 according to embodiment 3, like the split groove G4, also a split groove that splits the piezoelectric elements 3 adjacent to each other in the azimuth direction from each other extends from the end part of the acoustic matching layer 5 in the +Z-direction into the wiring board 2.

The ultrasound probe PB3 according to embodiment 3 is obtained by splitting the piezoelectric elements 3 and the acoustic matching layer 5 of the ultrasound probe PB1 of embodiment 1 in the azimuth direction with the split groove extending from the end part of the acoustic matching layer 5 in the +Z-direction into the wiring board 2, further splitting the piezoelectric elements 3 and the acoustic matching layer 5 in the elevation direction with the split groove G4, and forming the conductive layer 4 on the end part of the acoustic matching layer 5 in the +Z-direction, and the other configurations are the same as those of the ultrasound probe PB1 according to embodiment 1.

Even in the ultrasound probe PB3 having such a configuration, like the ultrasound probe PB1 of embodiment 1 and the ultrasound probe PB2 of embodiment 2, it is possible to generate a high-definition ultrasound image both in the shallow portion and in the deep portion by making the depth of the transmission focal point of an ultrasound beam electronically variable also in the elevation direction while suppressing an increase in the number of signal lines for driving.

The split groove G4 shown in FIG. 6 splits all the first acoustic matching layer 5A, the second acoustic matching layer 5B, and the third acoustic matching layer 5C of the acoustic matching layer 5, but without being limited thereto, can also be configured to extend from the inside of the wiring board 2 and the divisional element portions 31 to the middle of the acoustic matching layer 5 in the Z-direction. For example, the split groove G4 may be configured to split the first acoustic matching layer 5A and the second acoustic matching layer 5B of the acoustic matching layer 5 and not to split the third acoustic matching layer 5C, or the split groove G4 may be configured to split the first acoustic matching layer 5A of the acoustic matching layer 5 and not to split the second acoustic matching layer 5B and the third acoustic matching layer 5C.

### Embodiment 4

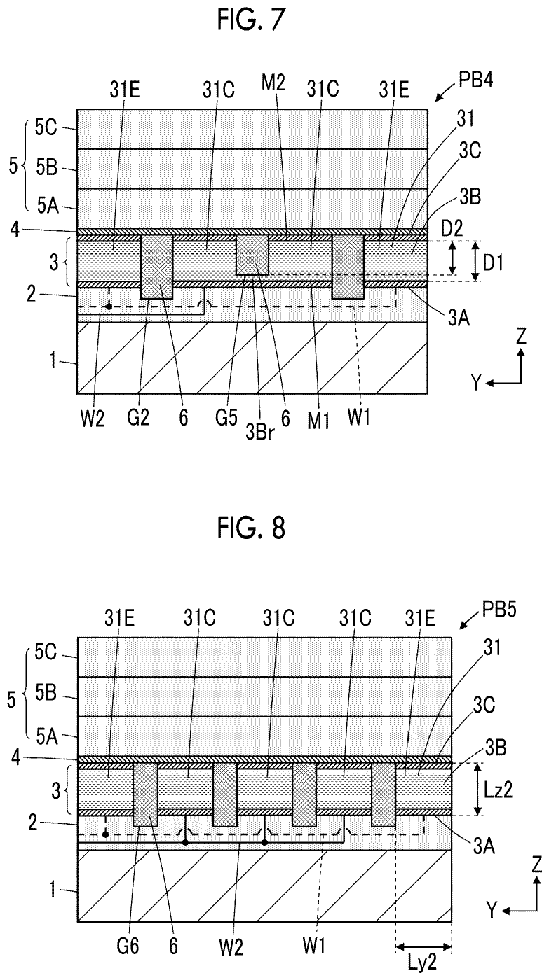

FIG. 7 shows a configuration of an ultrasound probe PB4 according to embodiment 4. In the ultrasound probe PB4, among the four divisional element portions 31 divided in the elevation direction, the two divisional element portions 31C positioned at the center are not completely split from each other, and a part of the piezoelectric portion 3B and the signal electrode layer 3A are connected to each other. A divisional groove G5 that extends from the boundary portion between the piezoelectric element 3 and the conductive layer 4 toward the wiring board 2 in the-Z-direction is formed between the two divisional element portions 31C.

The piezoelectric portion 3B of the divisional element portion 31C has a first surface M1 that is in contact with the signal electrode layer 3A and a second surface M2 that is in contact with the ground electrode layer 3C, and the divisional groove G5 extends in the piezoelectric portion 3B from the second surface M2 toward the first surface M1 and has a depth dimension D2 larger than 90% of a thickness D1 of the piezoelectric portion 3B in the Z-direction. That is, the two divisional element portions 31C are connected to each other via the signal electrode layer 3A that straddles these two divisional element portions 31C and a connecting portion 3Br that is positioned on a +Z-direction side of the signal electrode layer 3A and that remains as a part of the piezoelectric portion 3B. The signal electrode layer 3A common to the two divisional element portions 31C is connected to the conductive pattern W2 of the wiring board 2.

The ultrasound probe PB4 according to embodiment 4 is obtained by bringing the two divisional element portions 31C positioned at the center adjacent to each other in the elevation direction, among the four divisional element portions 31 in the ultrasound probe PB1 of embodiment 1, via the divisional groove G5 and connecting the two divisional element portions 31C to each other via the signal electrode layer 3A and the connecting portion 3Br, and the other configurations are the same as those of the ultrasound probe PB1 of embodiment 1.

The two divisional element portions 31C adjacent to each other in the elevation direction via the divisional groove G5 having the depth dimension D2 larger than 90% of the thickness D1 of the piezoelectric portion 3B in the Z-direction are connected to each other via the signal electrode layer 3A and the connecting portion 3Br, but are in a state of being split from each other in terms of mechanical vibration, and act in the same manner as the two divisional element portions 31C that are completely split as in the ultrasound probe PB1 of embodiment 1. Herein, the expression “split from each other in terms of mechanical vibration” means that, in a case where a voltage is applied to one divisional element portion 31C of the two divisional element portions 31C or vibration is generated by receiving an ultrasound echo, the vibration has almost no effect on the other divisional element portion 31C and is at a level at which the vibration cannot be measured.

For this reason, also in the ultrasound probe PB4 of embodiment 4, in a case where the aspect ratios R of the four divisional element portions 31 divided in the elevation direction are substantially the same and the difference dR between the maximum value Rmax and the minimum value Rmin of the aspect ratios R is within the range of 10% of the average value Rav of the aspect ratios R of the four divisional element portions 31, the frequency band shapes of the sensitivities in the four divisional element portions 31 are substantially the same.

Therefore, also in the ultrasound probe PB4 of embodiment 4, like the ultrasound probes PB1 to PB3 of embodiments 1 to 3, it is possible to generate a high-definition ultrasound image both in the shallow portion and in the deep portion by making the depth of the transmission focal point of an ultrasound beam electronically variable also in the elevation direction while suppressing an increase in the number of signal lines for driving.

In addition, in the ultrasound probe PB4 of embodiment 4, since the signal electrode layers 3A of the two divisional element portions 31C positioned at the center in the elevation direction are connected to each other and the signal electrode layers 3A are connected to the conductive pattern W2 of the wiring board 2, the wiring board 2 need only have one conductive pattern W2 for the two divisional element portions 31C, and the configuration of the wiring board 2 can be simplified.

### Embodiment 5

In the ultrasound probe PB1 of embodiment 1 shown in FIG. 2, each of the plurality of piezoelectric elements 3 arranged in an array in the azimuth direction is divided into the four divisional element portions 31 in the elevation direction, but the invention is not limited thereto.

FIG. 8 shows a configuration of an ultrasound probe PB5 according to embodiment 5. In the ultrasound probe PB5, each of the plurality of piezoelectric elements 3 arranged in the azimuth direction is divided into five divisional element portions 31 in the elevation direction. Each of the five divisional element portions 31 is split in the elevation direction via a split groove G6, and the split groove G6 is filled with the filler 6.

The five divisional element portions 31 have substantially the same aspect ratio R represented by a ratio of a thickness Lz2 in the Z-direction with respect to a length Ly2 in the elevation direction of each of the divisional element portions 31. Specifically, the difference dR between the maximum value Rmax and the minimum value Rmin of the aspect ratios R of the five divisional element portions 31 divided in the elevation direction is set to be within the range of 10% of the average value Rav of the aspect ratios R of the five divisional element portions 31.

Among the five divisional element portions 31 divided in the elevation direction, the signal electrode layers 3A of the divisional element portions 31E at two end parts disposed at both ends in the elevation direction are connected to one conductive pattern W1 of the wiring board 2, and the two divisional element portions 31E are configured to be simultaneously driven by a common drive voltage via the conductive pattern W1.

Further, among the five divisional element portions 31 divided in the elevation direction, the signal electrode layers 3A of all three divisional element portions 31C positioned at the center are connected to the conductive pattern W2 of the wiring board 2, and the three divisional element portions 31C are configured to be simultaneously driven by a common drive voltage via the conductive pattern W2.

The ultrasound probe PB5 according to embodiment 5 has the same configuration as that of the ultrasound probe PB1 of embodiment 1, except that the piezoelectric element 3 is divided into the five divisional element portions 31 in the elevation direction via the split grooves G6, and the signal electrode layers 3A of the three divisional element portions 31C positioned at the center are connected to the conductive pattern W2 of the wiring board 2.

Even in the ultrasound probe PB5 having such a configuration, like the ultrasound probes PB1 to PB4 of embodiments 1 to 4, it is possible to generate a high-definition ultrasound image both in the shallow portion and in the deep portion by making the depth of the transmission focal point of an ultrasound beam electronically variable also in the elevation direction while suppressing an increase in the number of signal lines for driving.

The piezoelectric element 3 can also be divided into five divisional element portions 31 in the elevation direction also in the ultrasound probes PB2 to PB4 of embodiments 2 to 4, as in embodiment 5.

In addition, each of the plurality of piezoelectric elements 3 arranged in the azimuth direction can also be divided into six or more and ten or less divisional element portions 31 in the elevation direction.

In embodiments 1 to 5, the four or five divisional element portions 31 divided in the elevation direction are classified into two groups consisting of two divisional element portions 31E disposed at both ends in the elevation direction and two or three divisional element portions 31C positioned at the center in the elevation direction, but the invention is not limited thereto. For example, the plurality of divisional element portions 31 divided in the elevation direction may be classified into three or more groups according to disposition positions in the elevation direction. In a group including at least two divisional element portions 31 that are positioned at the center in the elevation direction and that are adjacent to each other, the signal electrode layers 3A of the respective divisional element portions 31 may be to be electrically connected to each other and to be connected to conductive patterns of the wiring board 2 different from each other for each group.

### Explanation of References

1: backing material
2: wiring board
3: piezoelectric element
3A: signal electrode layer
3B: piezoelectric portion
3Br: connecting portion
3C: ground electrode layer
4: conductive layer
5: acoustic matching layer
5A: first acoustic matching layer
5B: second acoustic matching layer
5C: third acoustic matching layer
6: filler
PB1 to PB5: ultrasound probe
G1, G2, G3, G4, G6: split groove
31, 31E, 31C: divisional element portion
Ly1, Ly2: length
Lz1, Lz2, D1: thickness
C1, C2: curve
W1, W2: conductive pattern
P1: arrangement pitch
F1: groove width
G5: divisional groove
M1: first surface
M2: second surface
D2: depth dimension

What is claimed is:

1. An ultrasound probe in which a plurality of piezoelectric elements are arranged in an array along an azimuth direction on a backing material,
wherein each of the plurality of piezoelectric elements consists of a laminate in which a signal electrode layer, a piezoelectric portion, and a ground electrode layer are laminated in turn on a surface of the backing material,
each of the piezoelectric elements is divided into a plurality of divisional element portions, which are four or more and ten or less divisional element portions, in an elevation direction,
a difference between a maximum value and a minimum value of an aspect ratio represented by a ratio of a thickness with respect to a length of each of the plurality of divisional element portions in the elevation direction is within a range of 10% of an average value of aspect ratios of the plurality of divisional element portions,
an arrangement pitch of the plurality of divisional element portions in the elevation direction is larger than a wavelength of an ultrasound wave determined by a center frequency of the ultrasound probe, and
the signal electrode layers of at least two divisional element portions that are disposed at a center in the elevation direction and that are adjacent to each other, among the plurality of divisional element portions, are electrically connected to each other.

2. The ultrasound probe according to claim 1,
wherein a wiring board having a conductive pattern is disposed between the plurality of piezoelectric elements and the backing material, and
each of the signal electrode layers and the ground electrode layers of the plurality of piezoelectric elements is led out via the conductive pattern of the wiring board.

3. The ultrasound probe according to claim 2,
wherein each of the plurality of divisional element portions is split in the elevation direction via a split groove extending from the ground electrode layer to the signal electrode layer.

4. The ultrasound probe according to claim 3,
wherein the split groove extends into the wiring board.

5. The ultrasound probe according to claim 3,
wherein an acoustic matching layer is laminated on the ground electrode layers of the plurality of piezoelectric elements.

6. The ultrasound probe according to claim 4,
wherein an acoustic matching layer is laminated on the ground electrode layers of the plurality of piezoelectric elements.

7. The ultrasound probe according to claim 5,
wherein the split groove extends into the acoustic matching layer.

8. The ultrasound probe according to claim 6,
wherein the split groove extends into the acoustic matching layer.

9. The ultrasound probe according to claim 3,
wherein the split groove has a groove width of 10 μm or more and 30 μm or less in the elevation direction.

10. The ultrasound probe according to claim 4,
wherein the split groove has a groove width of 10 μm or more and 30 μm or less in the elevation direction.

11. The ultrasound probe according to claim 5,
wherein the split groove has a groove width of 10 μm or more and 30 μm or less in the elevation direction.

12. The ultrasound probe according to claim 6,
wherein the split groove has a groove width of 10 μm or more and 30 μm or less in the elevation direction.

13. The ultrasound probe according to claim 7,
wherein the split groove has a groove width of 10 μm or more and 30 μm or less in the elevation direction.

14. The ultrasound probe according to claim 8,
wherein the split groove has a groove width of 10 μm or more and 30 μm or less in the elevation direction.

15. The ultrasound probe according to claim 9,
wherein the split groove is filled with a filler.

16. The ultrasound probe according to claim 2,
wherein the at least two divisional element portions are split in the elevation direction in terms of mechanical vibration while each being connected to a part of the piezoelectric portion and the signal electrode layer.

17. The ultrasound probe according to claim 16,
wherein the piezoelectric portion has a first surface that is in contact with the signal electrode layer and a second surface that is in contact with the ground electrode layer, and
the piezoelectric portions of the at least two divisional element portions are adjacent to each other in the elevation direction via a divisional groove extending from the second surface toward the first surface.

18. The ultrasound probe according to claim 16,
wherein the divisional groove has a depth dimension larger than 90% of a thickness of the piezoelectric portion.

19. The ultrasound probe according to claim 16,
wherein the divisional groove has a groove width of 10 μm or more and 30 μm or less in the elevation direction.

20. The ultrasound probe according to claim 19,
wherein the divisional groove is filled with a filler.
